(19) United States
(12) Patent Application Publication
(10) Pub. No.: US 2021/0125696 A1
(43) Pub. Date: Apr. 29, 2021

(54) METHOD AND SYSTEM FOR PERSONALIZED HYPERTENSION TREATMENT

(71) Applicant: KONINKLIJKE PHILIPS N.V., EINDHOVEN (NL)

(72) Inventors: Jin LIU, Malden, MA (US); Michael PROKLE, Boston, MA (US)

(21) Appl. No.: 17/254,942

(22) PCT Filed: Jun. 26, 2019
(86) PCT No.: PCT/EP2019/067086

§ 371 Date: Dec. 22, 2020

Related U.S. Application Data
(60) Provisional application No. 62/691,021, filed on Jun. 28, 2018.

(51) Int. Cl. G16H 20/10; G06N 20/00; G16H 80/00

(52) U.S. Cl. CPC G16H 20/10; G16H 80/00; G06N 20/00

(57) ABSTRACT

A personalized hypertension treatment optimization system, including: a patient treatment model configured to receive patient data and blood pressure measurements and to produce a treatment recommendation including a type of medicine and dosage, wherein the patient treatment module includes an initial treatment machine learning model and an adjustment treatment machine learning module; a physician user interface configured to receive the treatment recommendation, patient data, and blood pressure measurement and to produce a treatment decision including a type of medicine and dosage based upon a physician input; and a patient user interface configured to receive a treatment decision and to display the treatment decision.

### TECHNICAL FIELD

Various exemplary embodiments disclosed herein relate generally to a method and system for personalized hypertension treatment.

### BACKGROUND

Although hypertension is very prevalent worldwide, the effective blood pressure control rate remains very low. Due to the complexity and high variety of the patient characteristics, it is unclear which type of antihypertensive medicine is most effective. Furthermore, finding the optimized dose of the medicine is another time-consuming process that is highly dependent on the compliance of the patient.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a personalized hypertension treatment optimization system, including: a patient treatment model configured to receive patient data and a blood pressure measurement and to produce a treatment recommendation including a type of medicine and dosage, wherein the patient treatment module includes an initial treatment machine learning model and an adjustment treatment machine learning model; a physician user interface configured to receive the treatment recommendation, patient data, and blood pressure measurement and to produce a treatment decision including a type of medicine and dosage based upon a physician input; and a patient user interface configured to receive a treatment decision and to display the treatment decision.

Various embodiments are described, wherein the initial treatment machine learning model includes a classification model to produce the type of medicine and a regression model to produce the dosage for a personalized initial treatment recommendation.

Various embodiments are described, wherein the initial treatment machine learning model is trained using lasso regression, principal components analysis, or random forest.

Various embodiments are described, wherein the adjustment treatment machine learning model includes a classification model to produce the adjusted type of medicine and a regression model to produce the adjusted dosage for a personalized adjustment treatment recommendation.

Various embodiments are described, wherein the adjustment treatment machine learning model is trained using machine learning models for longitudinal data analysis.

Various embodiments are described, wherein the patient treatment module determines if the patient's blood pressure is controlled and adjusting the treatment recommendation when patient's blood pressure is not controlled.

Various embodiments are described, wherein physician user interface includes a patient display, a medicine type display and dosage display, and a patient timeline display.

Various embodiments are described, wherein the physician user interface includes a reminder icon configured to produce reminders to the patient based upon physician input.

Various embodiments are described, wherein medicine type and dosage display are ranked by a predicted success rate for the recommended medicine.

Various embodiments are described, wherein the patient user interface is configured to receive manual data entry.

Various embodiments are described, wherein the patient user interface is configured to display one of a reminder notification, motivation notifications, success notifications, and education information.

Various embodiments are described, further including a machine learning module configured to produce: the initial treatment machine learning model based upon initial treatment training data; and the adjustment treatment machine learning model based upon adjustment treatment training data.

Various embodiments are described, wherein the initial treatment training data includes previous successful antihypertension treatment plan records with patient characteristics and antihypertension treatment guidelines.

Various embodiments are described, wherein the adjustment treatment training data includes previous successful antihypertension treatment plan records with longitudinal patient characteristics and antihypertension treatment guidelines.

Further various embodiments relate to a method for providing an optimized personalized hypertension treatment, including: receiving, by a patient treatment model, patient data and a blood pressure measurement; producing a treatment recommendation including a type of medicine and dosage; transmitting the treatment recommendation, patient data, and blood pressure measurement to a physician user interface; producing a treatment decision including a type of medicine and dosage based upon a physician input received via the physician user interface; and transmitting the treatment decision to patient user interface, wherein the patient treatment module includes an initial treatment machine learning model and an adjustment treatment machine learning model.

Various embodiments are described, wherein the initial treatment machine learning model includes a classification model to produce the type of medicine and a regression model to produce the dosage for a personalized initial treatment recommendation.

Various embodiments are described, wherein the initial treatment machine learning model is trained using lasso regression, principal components analysis, or random forest.

Various embodiments are described, wherein the adjustment treatment machine learning model includes a classification model to produce the adjusted type of medicine and a regression model to produce the adjusted dosage for a personalized adjustment treatment recommendation.

Various embodiments are described, wherein the adjustment treatment machine learning model is trained using machine learning models for longitudinal data analysis.

Various embodiments are described, further including determining if the patient's blood pressure is controlled and adjusting the treatment recommendation when patient's blood pressure is not controlled.

Various embodiments are described, further including transmitting reminders to the patient user interface based upon a physician reminder input.

### BRIEF DESCRIPTION OF THE DRAWINGS

In order to better understand various exemplary embodiments, reference is made to the accompanying drawings, wherein:

FIG. 1 illustrates a system for providing initial and adjusted hypertension treatment plans.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Embodiments described herein describe an automatic personalized hypertension treatment optimization solution that is trained based on previous patient data showing the outcomes of various hypertension treatments from a patient database to suggest both the best type of treatment and the best dose for initial treatment and ongoing treatment adjustments using patient characteristics and longitudinal blood pressure measurements.

Hypertension has high prevalence affecting 85.7 million people in the US (34.0%, US adults) and 972 million people worldwide. There have been a lot of antihypertensive drugs developed like diuretics, beta blockers (BB), Angiotensin converting enzyme (ACE) inhibitors (ACEI), Angiotensin II receptor blockers (ARB), calcium channel blockers (CCB), aldosterone antagonists (Aldo ANT), etc., based on different pharmacology.

Different types of drugs should be prescribed to different patients depending on their hypertension type and compelling indications. For heart failure, diuretics, BB, ACEI, ARB, and Aldo ANT have been shown to be effective. For postmyocardial infarction, BB, ACEI, and Aldo ANT have been shown to be effective. For high coronary disease risk patients, diuretics, BB, ACEI, and CCB have been shown to be effective. For diabetes, diuretics, BB, ACEI, ARB, and Aldo ANT have been shown to be effective. For chronic kidney disease, ACEI and ARB have been shown to be effective. For recurrent stroke prevention, diuretics and ACEI have been shown to be effective.

Current established guideline-based treatment may include the following. First the patient may try lifestyle modification where, for example, the patient improves their diet or increases their exercise. If these do not work, then the doctor may determine if there are compelling indications. If such compelling indications exist, such as for example, heart failure, postmyocardial infarction, high coronary disease risk, diabetes, chronic kidney disease, recurrent stroke prevention, or other conditions, then the doctor may select a drug treatment based upon compelling indication. If there are no compelling indications, the doctor may decide if the patient's hypertension is stage 1 or stage 2. In stage 1, thiazide-type diuretics are often prescribed or ACEI, ARB, BB, CCB, or some combination thereof may be chosen. In stage 2, typically a two-drug combination including thiazide-type diuretic along with one of an ACEI, ARB, BB, or CCB may be chosen. After a period of treatment, the doctor evaluates the patient's blood pressure and may then optimize dosages or add additional drugs or change drugs until the goal blood pressure is achieved.

Also, a more personalized approach could be more beneficial for hypertension treatment. Previous research has demonstrated the influence of genomic types (e.g., nephrosis gene variants, ALDH1A3 and CLIC5) and renin profiling on blood pressure response to antihypertension drugs.

Furthermore, patients need to follow up with their doctors monthly to make treatment adjustment until hypertension is effectively controlled. Both the most appropriate type and dose of drug vary with the patient characteristics and blood pressure lowering goal. Finding the optimized treatment is a time-consuming process requiring not only the cooperation of patients, but also physicians with rich hypertension treatment experience. The following guidelines provide recommendations for follow up regarding hypertension. For a patient with normal blood pressure, their blood pressure should be rechecked every two years. For a patient with prehypertension, their blood pressure should be rechecked every year. For a patient with stage 1 hypertension, the follow up is to confirm blood pressure within two months. For a patient with stage 2 hypertension, the follow up is to reevaluate the patient within one month, or for those with higher blood pressures (e.g., >180/110 mmHg), evaluate and treat immediately or within 1 week depending on clinical situation and complications.

A recent study found that less than half of hypertension patients were aware of this diagnosis. Among those who were aware, only a minority (32.5%) had their blood pressure properly controlled, although most of them (87.5%) were receiving hypertension medicines. The reasons for unsatisfactory hypertension control may include but are not limited to: 1) improper dose of antihypertension medicine; 2) improper antihypertension medicine type; 3) poor follow-up with doctor for treatment adjustment; and 4) poor patients' compliance to prescribed medicine.

This medicine type and dose adjustment process is inconvenient for the patient and demands a doctor with experience in treating hypertension, which may take a long time and numerous iterations to reach the blood pressure control goal. During the dose and type adjustment, patients are exposed to a high risk of stroke, heart disease, and kidney disease.

Therefore, developing an automated method to guide hypertension treatment including the medicine type and dose for both initial treatment and follow up adjustment plan will benefit hypertension patients.

An embodiment will be described below of a method using machine learning models (classification and regression) based on patient characteristics to provide recommendation of the best type and dose of antihypertensive medicine, as well as real-time treatment adjustment guidance based upon longitudinal blood pressure measurements.

Machine learning models may be developed based on patient data from a patient database to fit classification models and regression models with feature selection (e.g., Lasso) to determine the best medicine treatment plan for a patient. By using these models to determine the optimal medicine type and dose, for both initial treatment and later treatment adjustments, the patient's blood pressure may be more quickly and optimally controlled as compared to current methods based on doctor experience with tedious follow up visits.

The embodiments described herein include the following modules.

A module for training data extraction and database preparation that includes: data extraction from previous antihypertensive drug trails, guidelines, and patient database to get patient characteristics and successful treatment plan records; independent variables preparation for initial treatment, for example, patient age, gender, race, blood pressure, medical conditions (e.g., diabetes, pregnancy, renal failure), medicines in use and genomic information; and independent variables preparation for adjusted treatment, for example, longitudinal blood pressure measurements, previous medicine type and dose taken in addition to the initial treatment variables.

A module for machine learning model fitting that includes: a feature selection and classification model for the best antihypertensive medicine type(s) of initial treatment plan using initial treatment data prepared above including variables for initial treatment; a regression model with feature selection (e.g., Lasso) for the best antihypertensive medicine dose of initial treatment plan using initial treatment data prepared above including variables for initial treatment; a feature selection and classification model for the best antihypertensive medicine type(s) for an adjusted treatment plan using adjustment training data prepared above including variables for adjusted treatment; and a regression model with feature selection (e.g., Lasso) for the best antihypertensive medicine dose of adjusted treatment plan using adjustment training data prepared above including variables for adjusted treatment.

A module for automatic personalized patient hypertension treatment plan that: predicts the best type and dose of antihypertensive medicine for new patients, using the initial treatment plan classification model and regression model with new patient characteristics and blood pressure measurements; and makes treatment adjustment if the current treatment is not meeting the blood control goal, using the adjusted treatment plan classification model and regression model with both patient characteristics and longitudinal blood measurements.

Interactive user interfaces that include: a user interface for physicians and patients to enter patient characteristics and blood pressure measurements; a user interface for physicians to receive the treatment recommendation automatically with new patient data entered and then decide to accept or adjust based on the recommendation; a user interface for patients to receive the finalized treatment plan from physicians and to record the blood pressure manually or automatically using wearable devices and data collected through both interfaces to feed into the machine learning models and make necessary treatment plan adjustment suggestions in real-time.

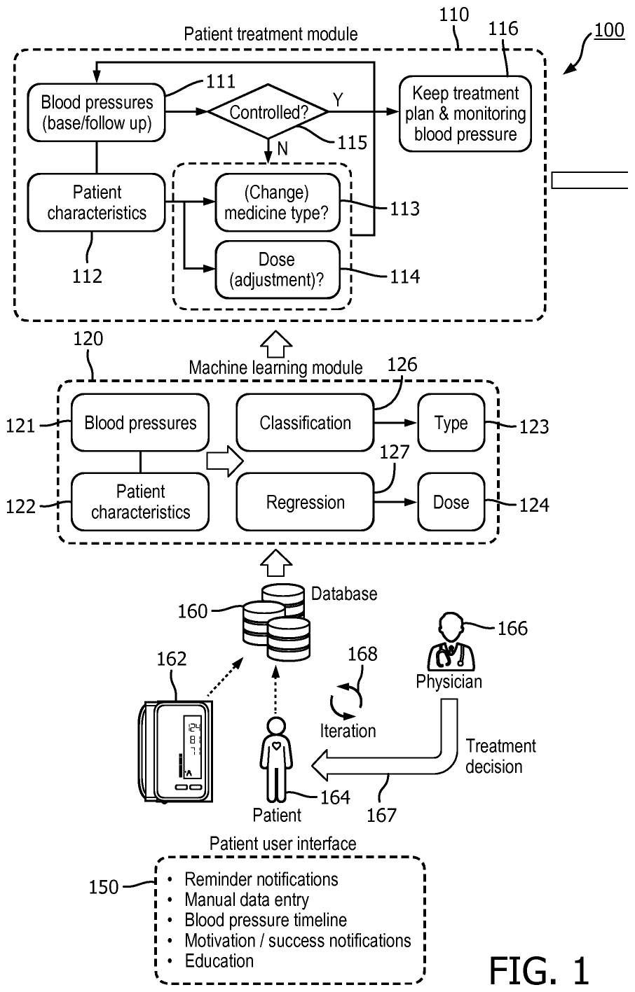

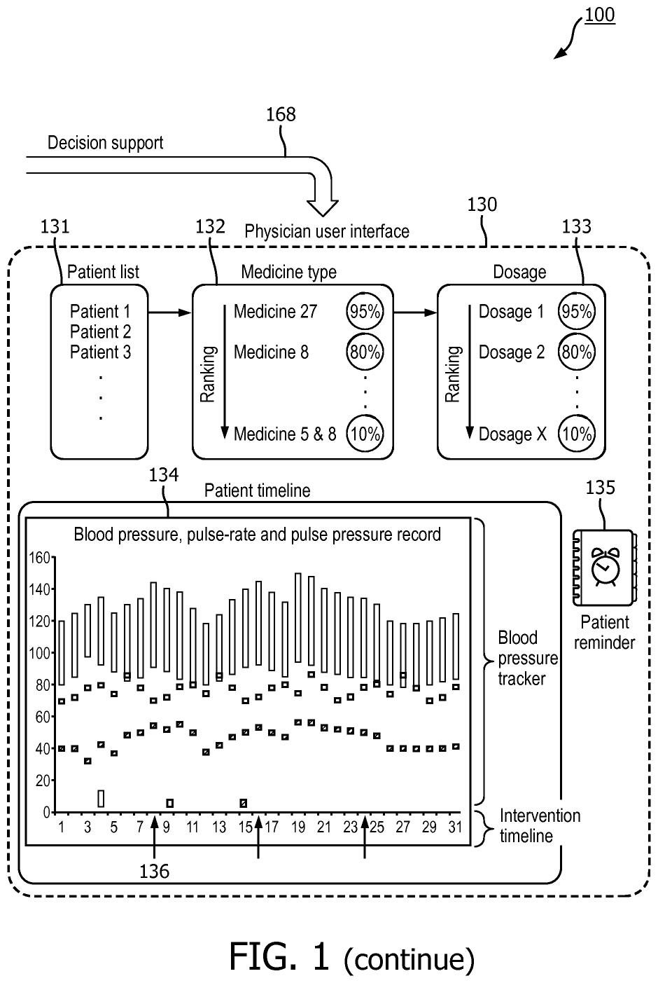

FIG. 1 illustrates a system for providing initial and adjusted hypertension treatment plans. The blood pressure treatment system 100 may include a patient treatment module 110, a machine learning module 120, a physician user interface 130, and a patient user interface 150.

A patient database 160 collects data from patients 164 and patient monitoring equipment 162. Such data may include, for example, patient age, gender, race, blood pressure, medical conditions (e.g., diabetes, pregnancy, renal failure), medicines in use, genomic information, etc. The database 160 may also include data from previous antihypertensive drug trials and guidelines.

The machine learning module 120 may include a module as described above for training data extraction and database preparation. This module extracts training data from the database 160 that may include: data extraction from previous antihypertensive drug trails, guidelines, and patient database to get patient characteristics and successful treatment plan records; independent variables preparation for initial treatment, for example, patient age, gender, race, blood pressure, medical conditions (e.g., diabetes, pregnancy, renal failure), medicines in use and genomic information; and independent variables preparation for adjusted treatment, for example, longitudinal blood pressure measurements, previous medicine type and dose taken in addition to the initial treatment variables. The extracted training data may include initial treatment training data that is used for training a model for producing an initial treatment and adjustment treatment training data that is used for training a model for producing an adjustment treatment.

The machine learning module 120 develops two models: an initial treatment model and an adjustment treatment model. The initial treatment model receives patient input such as blood pressure 121 and other patient characteristics 122 and determines an initial treatment plan for the patient including the type of medicine(s) 123 and dosage 124. Previous antihypertensive treatment guidelines may be considered to create rules for the initial treatment model. This model may be trained using the initial treatment training data. The initial treatment model includes a classification model 126 and a regression model 127. The classification model 126 includes feature selection and classification. Training the classification model includes determining based upon the initial treatment training data, which are the best features for predicting what type of medicine(s) to use to initially treat the patient. Various types of models for classification may be used including, for example, multiple linear logistic regression, random forest, support vector machines (SVM), and K nearest neighbor classifier (KNN). The regression model 127 determines what the initial dosage of the selected medicine should be based upon patient input data. Training the regression model includes determining based upon the initial training data, which are the best features for predicting what dosage of the medicine(s) to use to initially treat the patient. Various types of models for regression may be used including, for example, multiple linear regression and random forest. Alternatively, for either classification or regression, an integrated model may be used instead by using regularization/penalization or dimension reduction methods without feature pre-selection. Such methods include, for example, lasso logistic regression and principal components analysis. Such a model is trained using the initial treatment training data and produces a model that will produce both the type of medicine(s) to be used as well as the dosage.

The adjustment treatment model receives patient input such as longitudinal blood pressure measurements 121 and other patient characteristics 122 and determines an adjustment treatment plan for the patient including the type of medicine(s) 123 and dosage 124. This model may be trained using the adjustment treatment training data. The adjustment treatment model includes a classification model 126 and a regression model 127. The classification model 126 includes feature selection and classification. Training the classification model includes determining based upon the adjustment treatment training data, which are the best features for predicting what type of medicine to use, which might include changing which medicine(s) to use to treat the patient going forward. Various types of models for classification may be used including, for example, multiple linear logistic regression, random forest, support vector machines (SVM), K nearest neighbor classifier (KNN), and longitudinal machine learning methods such as recursive neural network (RNN), long short-term memory (LSTM), and penalized linear mixed effects models. The regression model 127 determines what the adjusted dosage of the selected medicine(s) should be based upon patient input data. Training the regression model includes determining based upon the adjustment training data, which are the best features for predicting what dosage of the medicine(s) to use to treat the patient going forward. Various types of models for regression may be used including, for example, multiple linear regression and random forest. Alternatively, for either classification or regression, an integrated model may be used instead by using regularization/penalization or dimension reduction methods without feature pre-selection. Such methods include, for example, lasso logistic regression and principal components analysis. Such a model is trained using the adjustment treatment training data and produces a model that will produce both the type of medicine(s) to be used as well as the dosage.

The patient treatment module 110 uses the initial treatment model and the adjustment treatment model to provide treatment recommendations for new patients. The patient treatment module receives blood pressure measurements 111 and patient characteristics 112 for a specific patient. For a patient's beginning treatment, the initial treatment model is used to prescribe a medicine type 113 and the dosage 114. Then after the patient has been treated additional blood pressure measurements 111 are taken. The patient treatment module 110 then determines if the patient's blood pressure has been controlled 115. If so, then the treatment plan is kept and the patient's blood pressure continues to be monitored 116. If the patient's blood pressure is not controlled, the patient treatment module 110 uses the adjustment treatment module to change the type of medicine(s) used to treat the patient 113 and/or the dosage of medicine(s) taken by the patient 114. It is noted, that the adjustment model may first change dosages of an existing medicine before changing the medicine type, but the adjustment treatment model will provide a treatment recommendation based upon what was learned from the adjustment training data. The patient treatment module provides a treatment recommendation via decision support 168 to the physician user interface.

The physician's user interface 130 provides a user interface that the physician uses to provide treatment to a number of patients. The physician user interface may include a patient list display 131, medicine type(s) display 132, dosage display 133, a patient timeline display 134, and patient reminders icon 135.

The patient list display 131 presents a list of patients currently under the physician's care. The list may include a list of names or other identifying information. The list may be arranged in various orders, and such listing order may be configurable. For example, the list may be ordered based upon how long it has been since the patient was last evaluated or reviewed by the physician. Also, the list may be prioritized based upon the need for physician review. For example, if a patient shows a sudden increase in recent blood pressure measurements, then that patient may be prioritized for review. Further, patients in the patient list may be annotated in some way when the patient urgently requires attention. When the physician selects a specific patient, then patient specific information such as medicine type, dosage, patient timeline, and patient reminder are shown in the related displays.

The medicine type display 132 may show various information related to medicines recommended to and/or used by the patient. Depending up information from the patient treatment module 110, recommendations for an initial or change in the type of medicine to prescribe may be shown. Multiple recommendations may be shown in order of predicted success rate based upon current patient information. The success rate may be shown beside recommended medicine type to facilitate physicians' decision. The type of medicine may include a single medicine or combinations of medicines. Also, the current medicine in use may be shown as well as medicines previously used.

The dosage display 133 displays dosage information related to the medicine types shown in the medicine type display 132. For each medicine shown in the medicine type display 132, a related dosage may be shown in the dosage display 133. These dosage amounts may be recommended, current, or historic dosage amounts based upon the corresponding information shown in the medicine type display 132. Further, if a physician is looking to change the medicine type 132, then when the recommended medicine type is selected, a list of recommended dosages for the patient is presented in order of preference based upon output of the patient treatment module 110. Further, using the medicine type display 132, the physician may select, accept, and/or modify the medicine type and dosages to be prescribed to the patent.

The patient timeline display 134 may display a variety of historical patient information. For example, blood pressure, pulse rate, and pulse pressure values may be plotted over time and displayed. Further, specific interventions 136 may be annotated on the plot to show for example where changes in treatment occurred, doctor's visits occurred, or other interventions occurred. The patient timeline information may include other sorts and combinations of information as well. A variety of different patient timeline display types may be predefined, and the user may also be able to customize what patient timeline information is shown. The patient timeline display provides the physician with a variety of information that helps the physician make treatment decisions for the patient.

The patient reminder icon 135 that the physician may use to send a reminder to the patient. When the physician selects the patient reminder icon, another display may be presented indicating the types reminders that should be sent to the patient and when. Such reminders may include, for example, reminding the patient to record blood pressure information more consistently, to schedule a follow up appointment, to strive to make certain lifestyle changes, or to carry out some other sort of task. Such reminders may be sent out using a variety of methods, including mail, email, phone call, text message, etc.

The physician 166 may use the physician user interface 130 and the patient treatment module 110 to provide a treatment decision 167 to the patient 164. Such treatment decision may be an initial indication of the need for blood pressure medication and what type of medicine(s) and the dosage. If the patient 164, is being currently treated and the physician 166 determines a change in treatment is needed, then the updated treatment plan is what is communicated in the treatment decision 167. The treatment decision may include other courses of action and information as well. For example, in addition to treatment, lifestyle changes may be recommended to the patient 164. Such treatment review and changes to the treatment plan may be iterated 168 as needed to achieve and maintain a desired blood pressure level for the patient 164.

The patient user interface 150 may be used by the patient to receive reminder notifications, motivation and/or success motivations, and education regarding their condition. Also, the patient user interface may allow for the patient to manually enter data of various types, for example, blood pressure, patient characteristics, how the patient is feeling, any complications or side-effects, etc. Also, historic data such as a blood pressure timeline may be shown. Such data presented may be the same as or a part of that presented to the physician in the patient timeline display 134. A motivation/success notification system may be implemented, for example, by giving a “star” if the patient takes medicine on time and/or has blood pressure controlled for that day. Likewise, predefined data display types may be available as well as the option for the patient to customize what data is presented.

The blood pressure treatment system 100 provides the patient and physician tools to better treat the patient's hypertension. Patient treatment options are developed based upon machine learning models trained on patient data. Such models may provide initial treatment recommendations as well as changes to treatment plans to achieve the desired blood pressure level. As these models are based upon prior successful treatment plans, the physician can more quickly converge on a successful treatment plan for the patient. The blood pressure treatment system 100 improves the ability to monitor and treat the patient's hypertension and increases the chance of successfully reducing the patient's blood pressure. The patient user interface allows the patient to enter data related to their condition. In some situations, as the patient uses devices to measure blood pressure, such data may automatically be recorded for making further treatment evaluations. This reduces the need for the patient to make an office visit, and thus improve the chances of successfully treating the patient's hypertension. Further, the physician is able to use the patient treatment module and physician user interface to evaluate the patient and make further treatment recommendations without the need for an office visit. This also improves the chances of achieving a successful treatment plan. These and other various features of the embodiments described above result in a technological improvement and advancement over existing hypertension treatment methods and systems.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems.

For example, the hypertension treatment system may be implemented as software on a server, a specific computer, on a cloud computing, or other computing platform. This software then provides the physician user interface and the patient user interface via, for example, a web-based interface, stand-alone software, other methods on the devices used by the physician and patient. The devices used by the physician may be any type of computing device, for example, desktop computers, laptop computers, tablets, smart phones, smart watches and wearable devices, etc., capable of interacting with or hosting the software implementing the hypertension treatment system. The devices used by the patient may be any type of computing device, for example, desktop computers, laptop computers, tablets, smart phones, smart watches and wearable devices, etc., capable of interacting with the software implementing the hypertension treatment system.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A personalized hypertension treatment optimization system, comprising:
a patient treatment model configured to receive patient data and a blood pressure measurement and to produce a treatment recommendation including a type of medicine and dosage, wherein the patient treatment module includes an initial treatment machine learning model and an adjustment treatment machine learning model;
a physician user interface configured to receive the treatment recommendation, patient data, and blood pressure measurement and to produce a treatment decision including a type of medicine and dosage based upon a physician input; and
a patient user interface configured to receive a treatment decision and to display the treatment decision.

2. The system of claim 1, wherein the initial treatment machine learning model includes a classification model to produce the type of medicine and a regression model to produce the dosage for a personalized initial treatment recommendation.

3. The system of claim 1, wherein the initial treatment machine learning model is trained using lasso regression, principal components analysis, or random forest.

4. The system of claim 1, wherein the adjustment treatment machine learning model includes a classification model to produce the adjusted type of medicine and a regression model to produce the adjusted dosage for a personalized adjustment treatment recommendation.

5. The system of claim 1, wherein the adjustment treatment machine learning model is trained using machine learning models for longitudinal data analysis.

6. The system of claim 1, wherein the patient treatment module determines if the patient's blood pressure is controlled and adjusting the treatment recommendation when patient's blood pressure is not controlled.

7. The system of claim 1, wherein physician user interface includes a patient display, a medicine type display and dosage display, and a patient timeline display.

8. The system of claim 7, wherein the physician user interface includes a reminder icon configured to produce reminders to the patient based upon physician input.

9. The system of claim 7, wherein medicine type and dosage display are ranked by a predicted success rate for the recommended medicine.

10. The system of claim 1, wherein the patient user interface is configured to receive manual data entry.

11. The system of claim 1, wherein the patient user interface is configured to display one of a reminder notification, motivation notifications, success notifications, and education information.

12. The system of claim 1, further comprising a machine learning module configured to produce:
the initial treatment machine learning model based upon initial treatment training data; and
the adjustment treatment machine learning model based upon adjustment treatment training data.

13. The system of claim 12, wherein the initial treatment training data includes previous successful antihypertension treatment plan records with patient characteristics and antihypertension treatment guidelines.

14. The system of claim 12, wherein the adjustment treatment training data includes previous successful antihypertension treatment plan records with longitudinal patient characteristics and antihypertension treatment guidelines.

15. A method for providing an optimized personalized hypertension treatment, comprising:
receiving, by a patient treatment model, patient data and a blood pressure measurement;
producing a treatment recommendation including a type of medicine and dosage;
transmitting the treatment recommendation, patient data, and blood pressure measurement to a physician user interface;
producing a treatment decision including a type of medicine and dosage based upon a physician input received via the physician user interface; and
transmitting the treatment decision to patient user interface,
wherein the patient treatment module includes an initial treatment machine learning model and an adjustment treatment machine learning model.

16. The method of claim 15, wherein the initial treatment machine learning model includes a classification model to produce the type of medicine and a regression model to produce the dosage for a personalized initial treatment recommendation.

17. The method of claim 15, wherein the initial treatment machine learning model is trained using lasso regression, principal components analysis, or random forest.

18. The method of claim 15, wherein the adjustment treatment machine learning model includes a classification model to produce the adjusted type of medicine and a regression model to produce the adjusted dosage for a personalized adjustment treatment recommendation.

19. The method of claim 15, wherein the adjustment treatment machine learning model is trained using machine learning models for longitudinal data analysis.

20. The s method of claim 15, further comprising determining if the patient's blood pressure is controlled and adjusting the treatment recommendation when patient's blood pressure is not controlled.

21. The method of claim 15, further comprising transmitting reminders to the patient user interface based upon a physician reminder input.
